(19) United States
(12) Patent Application Publication
(10) Pub. No.: US 2023/0381156 A1
(43) Pub. Date: Nov. 30, 2023

(54) TREATMENT OF BREAST CANCER USING COMBINATION THERAPIES COMPRISING GDC-9545 AND IPATASERTIB

(71) Applicant: Genentech, Inc., South San Francisco, CA (US)

(72) Inventors: Ciara METCALFE, Kensington, CA (US); Kui LIN, Foster City, CA (US); Marc Antoine HAFNER, Menlo Park, CA (US)

(73) Assignee: Genentech, Inc., South San Francisco, CA (US)

(21) Appl. No.: 18/449,807

(22) Filed: Aug. 15, 2023

Related U.S. Application Data
(63) Continuation of application No. PCT/US2022/016254, filed on Feb. 14, 2022.
(60) Provisional application No. 63/149,947, filed on Feb. 16, 2021.

(51) Int. Cl. A61K 31/437; A61K 31/517

(52) U.S. Cl. CPC A61K 31/437; A61K 31/517

(57) ABSTRACT

Provided herein are combination therapies comprising GDC-9545 and ipatasertib for treating locally advanced breast cancer or metastatic breast cancer.

### CROSS-REFERENCE TO RELATED APPLICATIONS

This application is a Continuation of International Application No. PCT/US2022/016254, filed Feb. 14, 2022, which claims the benefit of U.S. Provisional Patent Application No. 63/149,947, filed 16 Feb. 2021, which is incorporated herein by reference in its entirety and for all purposes.

### FIELD OF THE INVENTION

Provided herein are combination therapies comprising a GDC-9545 or a pharmaceutically acceptable salt thereof) and ipatasertib or a pharmaceutically acceptable salt thereof for the treatment of breast cancers.

### BACKGROUND

Breast cancer is the most frequent cancer diagnosed in women, with an estimated global incidence of 2.1 million new cases reported in 2018 (Bray et al. C A Canver J Clin 2018; 68:394-424). Breast cancer accounts for approximately 12% (approximately 627,000 cases) of all cancer deaths. Breast cancer mortality rates differ by geographical region, with more favorable survival rates observed in more developed regions of the world (Id).

Initial treatment for breast cancer is often guided by the presence of molecular markers found on breast cancer cells. These markers are used to identify breast cancer subtypes and to assist in the development of treatments based on presence of tumor hormone receptor content (estrogen receptor [ER]/progesterone receptor [PR]). Hormone receptor-positive (HR+) breast cancers with estrogen receptor—alpha (ER-α)-expression constitute 70% of all invasive breast cancers. PR expression in the tumor is another marker of ER-α signaling. Endocrine agents are the standard-of-care treatment used to downregulate ER signaling for HR+ breast cancers.

Human epidermal growth factor receptor 2 (HER2) is a transmembrane receptor tyrosine kinase that is amplified or overexpressed in 20% of breast cancers. HER2-positive breast cancer treatment regimens include HER2-directed therapies (anti-HER2 antibodies and tyrosine kinase inhibitors).

Not all HR+ breast cancers respond optimally to ET. Mechanisms that can lead to primary and/or secondary hormonal resistance in HR+ breast cancer include a decrease or loss of hormone receptor expression or an upregulation of growth factor signaling pathways, such as the epidermal growth factor receptor or HER2, the MAPK, or the PI3K/Akt/mTOR pathways. Recently, mutations in the gene that encodes estrogen receptor (ESR1) have been identified in metastatic ER-positive (ER+) tumors and are associated with resistance to anti-estrogen therapies.

Accordingly, there is a pressing need for clinically active agents for treatment of relapsed or resistant ER-positive breast cancer.

### SUMMARY

Provided herein are solutions to the problems above and other problems in the art.

The present embodiments can be understood more fully by reference to the detailed description and examples, which are intended to exemplify non-limiting embodiments.

### BRIEF DESCRIPTION OF FIGURES

FIG. 1 depicts the combination benefits of GDC-9545 and ipatasertib in aggregate across various HR+ cell lines. Systematic increased efficacy is shown across 4 out of the 9 lines tested.

FIG. 2 depicts the synergistic response of GDC-9545 and ipatasertib in aggregate across various HR+ cell lines as excess over Bliss index. Systematic increased efficacy is shown across 4 of the 9 lines tested.

FIG. 3 depicts the single dose response of GDC-9545 and GDC-0068 with HAS excess <−0.1. Combination benefits are shown in 5 of the 9 lines tested. GDC-0068=ipatasertib.

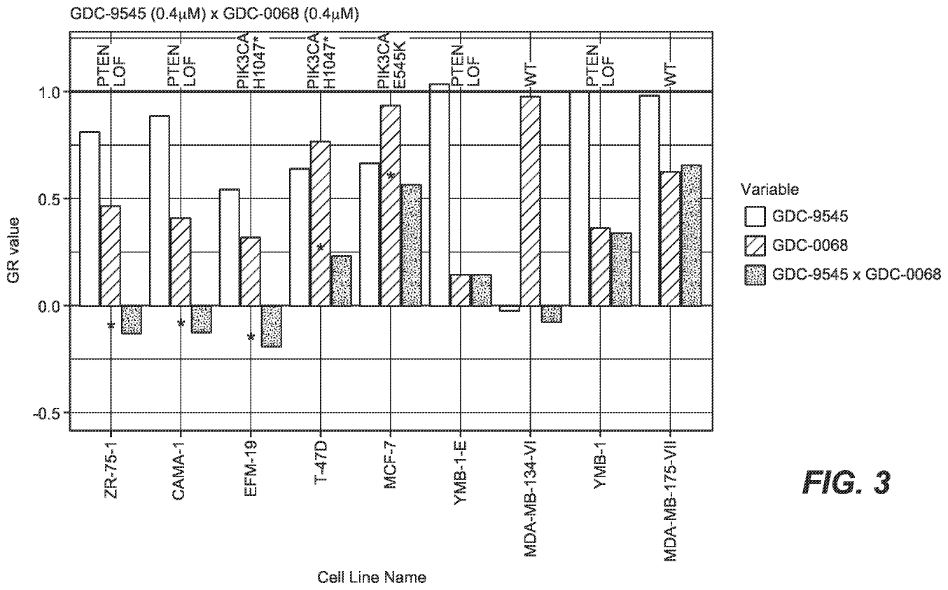

### DETAILED DESCRIPTION

Unless defined otherwise, all technical and scientific terms used herein have the same meaning as commonly understood by those of ordinary skill in the art to which the invention belongs. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention.

The following definitions are provided to facilitate understanding of certain terms used frequently herein and are not meant to limit the scope of the present disclosure. All references referred to herein are incorporated by reference in their entirety.

As used herein, and unless otherwise specified, the terms “about” and “approximately,” when referring to doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. The equivalent dose, amount, or weight percent can be within 30%, 20%, 15%, 10%, 5%, 1%, or less of the specified dose, amount, or weight percent.

“GDC-9545” refers to a compound having the structure:

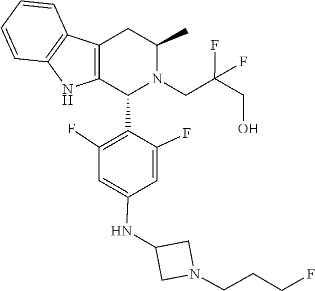 [SMILES: C[C@@H]1Cc2c([nH]c3ccccc23)[C@@H](c2c(F)cc(NC3CN(CCCF)C3)cc2F)N1CC(F)(F)CO]

having the chemical name 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol, including a pharmaceutically acceptable salt thereof. In one embodiment, GDC-9545 is a tartrate salt. “GDC-9545” as used herein refers to free base and pharmaceutically acceptable salts of GDC-9545 including a tartrate salt thereof. GDC-9545 is also known as giredestrant.

“Ipatasertib” refers to a compound having the structure:

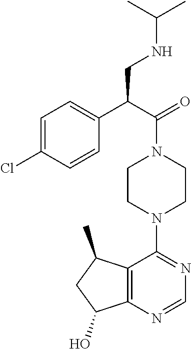 [SMILES: CC(C)NC[C@@H](C(=O)N1CCN(c2ncnc3c2[C@H](C)C[C@H]3O)CC1)c1ccc(Cl)cc1]

having the chemical name (S)-2-(4-chlorophenyl)-1-(4-((5R,7R)-7-hydroxy-5-methyl-6,7-dihydro-5H-cyclopenta[d]pyrimidin-4-Apiperazin-1-yl)-3-(isopropylamino)propan-1-one, including a pharmaceutically acceptable salt thereof. In one embodiment, ipatasertib is an amorphous mono-HCI salt. “Ipatasertib” as used herein refers to free base and pharmaceutically acceptable salts of ipatasertib including a mono-HCI salt thereof.

“Overall survival” or “OS” refers to the time from enrollment to death from any

cause.

“Objective Response” refers to a complete response or partial response, as determined by an investigator according to RECIST v1.1.

“Objective response rate” or “ORR” refers the percentage of patients with a confirmed complete response or partial response on two consecutive occasions 4 weeks apart, as determined by the investigator according to RECIST v1.1.

“Time to progression” or “TTP” refers to the time from randomization until objective tumor progression.

“Duration of response” or “DOR” refers to the time from the first occurrence of a documented objective response to disease progression, as determined by the investigator according to RECIST v1.1, or death from any cause, whichever occurs first.

“Progression free survival” or “PFS” refers to the time from enrollment to the date of the first recorded occurrence of disease progression, as determined by the investigator using RECIST v1.1 or death from any cause, whichever occurs first.

“Disease Control Rate” or “DCR” refers to the proportion of patients with stable disease for at least 12 weeks or a CR or PR as determined by the investigator using RECIST v1.1.

“Clinical benefit rate” or “CBR” refers to the percentage of patients with stable disease for at least 24 weeks or with confirmed complete or partial response, as determined by the investigator according to RECIST v1.1.

“Complete response” or “CR” refers to the disappearance of all target lesions and non-target lesions and (if applicable) normalization of tumor marker level.

“Partial response” or “non-CR/Non-PD” refers to persistence of one or more non-target lesions and/or (if applicable) maintenance of tumor marker level above the normal limits. A PR can also refer to 30% decrease in sum of diameters of target lesions, in the absence of CR, new lesions, and unequivocal progression in non-target lesions.

“Progressive disease” or “PD” refers to 20% increase in sum of diameters of target lesions, unequivocal progression in non-target lesions, and/or appearance of new lesions.

“Stable disease” or “SD” refers to neither sufficient shrinkage to qualify for CR or PR nor sufficient increase growth of tumor to qualify for PD.

The term “locally advanced breast cancer” refers to cancer that has spread from where it started in the breast to nearby tissue or lymph nodes, but not to other parts of the body.

The term “metastatic breast cancer” refers to cancer that has spread from the breast to other parts of the body, such as the bones, liver, lungs, or brain. Metastatic breast cancer may also be referred to as stage IV breast cancer.

The term “treatment” refers to clinical intervention designed to alter the natural course of the patient or cell being treated during the course of clinical pathology. Desirable effects of treatment include decreasing the rate of disease progression, ameliorating or palliating the disease state, and remission or improved prognosis. For example, a patient is successfully “treated” if one or more symptoms associated with a breast cancer described herein are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of patients.

The term “delaying progression” of a disease refers to deferring, hindering, slowing, retarding, stabilizing, and/or postponing development of a breast cancer described herein. This delay can be of varying lengths of time, depending on the history of the cancer and/or patient being treated. As is evident to one skilled in the art, a sufficient or significant delay can, in effect, encompass prevention, in that the patient does not develop cancer.

An “effective amount” is at least the minimum amount required to effect a measurable improvement or prevention of a breast cancer described herein. An effective amount herein may vary according to factors such as the disease state, age, sex, and weight of the patient, and the ability of the agent to elicit a desired response in the patient. An effective amount is also one in which any toxic or detrimental effects of the treatment are outweighed by the therapeutically beneficial effects. Beneficial or desired results include results such as eliminating or reducing the risk, lessening the severity, delaying the onset of the disease (including biochemical, histological and/or behavioral symptoms of the disease, its complications and intermediate pathological phenotypes presenting during development of the disease), decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In some embodiments, an effective amount of the drug may have the effect in reducing the number of cancer cells; reducing the tumor size; inhibiting (i.e., slow or stop) cancer cell infiltration into peripheral organs; inhibit (i.e., slow or stop) tumor metastasis; inhibiting (i.e., slow or stop) tumor growth; and/or relieving one or more of the symptoms associated with the disorder. An effective amount can be administered in one or more administrations. An effective amount of drug, compound, pharmaceutical composition, or combination therapy described herein can be an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition, or combination therapy. Thus, an “effective amount” may be considered in the context of administering one or more therapeutic agents, and a single agent may be considered to be given in an effective amount if, in conjunction with one or more other agents, a desirable result may be or is achieved.

An “E2-repressed score” as used herein, refers to a numerical value that reflects an aggregated expression level of a predetermined set of genes whose repression is reflective of estrogen receptor (ER) pathway activity.

An “E2-induced score” as used herein, refers to a numerical value that reflects an aggregated expression level of a predetermined set of genes whose induction is reflective of estrogen receptor (ER) pathway activity.

An “ER pathway activity score” as used herein, refers to a numerical value that reflects mathematical difference between the E2-induced score and the E2-repressed score.

An “administration period” or “cycle” refers to a period of time comprising administration of one or more agents described herein (i.e. GDC-9545 or a pharmaceutically acceptable salt thereof or ipatasertib or a pharmaceutically acceptable salt thereof) and an optional period of time comprising no administration of one or more of the agents described herein. For example, a cycle can be 28 days in total length and include administration of one or more agents for 21 days and a rest period of 7 days. A “rest period” refers to a period of time where at least one of the agents described herein (e.g. GDC-9545 or a pharmaceutically acceptable salt thereof or ipatasertib or a pharmaceutically acceptable salt thereof) are not administered. In one embodiment, a rest period refers to a period of time where none of the agents described herein (e.g. GDC-9545 or a pharmaceutically acceptable salt thereof or ipatasertib or a pharmaceutically acceptable salt thereof) are administered. A rest period as provided herein can in some instances include administration of another agent that is not GDC-9545 or a pharmaceutically acceptable salt thereof or ipatasertib or a pharmaceutically acceptable salt thereof. In such instances, administration of another agent during a rest period should not interfere or detriment administration of an agent described herein.

A “dosing regimen” refers to a period of administration of the agents described herein comprising one or more cycles, where each cycle can include administration of the agents described herein at different times or in different amounts.

“QD” refers to administration of a compound once daily.

“PO” refers to oral administration of an agent described herein.

A graded adverse event refers to the severity grading scale as established for by NCI CTCAE. In one embodiment, the adverse event is graded in accordance with the table below.

| Grade | Severity |
|---|---|
| 1 | Mild; asymptomatic or mild symptoms; clinical or diagnostic |
| | observations only; or intervention not indicated |
| 2 | Moderate; minimal, local, or non-invasive intervention indicated; |
| | or limiting age-appropriate instrumental activities of daily living ^{a} |
| 3 | Severe or medically significant, but not immediately life- |
| | threatening; hospitalization or prolongation of hospitalization |
| | indicated; disabling; or limiting self-care activities of daily |
| | living ^{b, c} |
| 4 | Life-threatening consequences or urgent intervention indicated ^{d} |
| 5 | Death related to adverse event ^{d} |

Combination Therapies

Provided herein are combination therapies comprising GDC-9545 or a pharmaceutically acceptable salt thereof (e.g. GDC-9545.tartrate) and ipatasertib or a pharmaceutically acceptable salt thereof (e.g. ipatasertib mono-HCI). In one embodiment, the combination therapies described herein are useful in the treatment of certain types of breast cancer as described herein. In one aspect provided herein is a combination therapy comprising GDC-9545 or a pharmaceutically acceptable salt thereof administered QD on days 1-28 of a first 28-day cycle and ipatasertib or a pharmaceutically acceptable salt thereof administered QD on days 1-21 of the first 28-day cycle.

In one embodiment of the combination therapy described herein GDC-9545 or a pharmaceutically acceptable salt thereof is administered as a fixed dose QD administration. In one embodiment, the administration is oral (PO), where GDC-9545 or a pharmaceutically acceptable salt thereof is formulated as a tablet or capsule. In one embodiment, GDC-9545 or a pharmaceutically acceptable salt thereof is administered at an amount of about 1 mg-100 mg, 1 mg-50 mg, 1 mg-30 mg, 10 mg-100 mg, 10 mg-50 mg, or 10 mg-30 mg QD. In another embodiment, GDC-9545 or a pharmaceutically acceptable salt thereof is administered at an amount of about 1, 5, 10, 15, 20, 25, 30, 50, or 100 mg. In still another embodiment, GDC-9545 or a pharmaceutically acceptable salt thereof is administered at an amount of about 30, 50, or 100 mg. In still another embodiment, GDC-9545 or a pharmaceutically acceptable salt thereof is administered at an amount of 30 mg.

In one embodiment of the combination therapy described herein, ipatasertib is administered at an amount of 400 mg. Such administration can be in a single dose (i.e. a single or multiple pills). In one embodiment, the dose of ipatasertib is reduced to 300 mg or 200 mg when a patient described herein experiences an adverse event. Ipatasertib can be administered PO QD as described herein.

The combination therapies described herein can be provided as a kit comprising one or more of the agents for administration. In one embodiment, the kit includes GDC-9545 or a pharmaceutically acceptable salt thereof for administration in combination with ipatasertib as described herein. In another embodiment, the kit includes GDC-9545 or a pharmaceutically acceptable salt thereof packaged together with ipatasertib, where the kit comprises separate formulated dosages of each agent. In still another embodiment, the kit includes GDC-9545 or a pharmaceutically acceptable salt thereof co-formulated with ipatasertib.

In one embodiment, the agents of the combination therapy described herein are supplied in a kit in a form ready for administration or, for example, as a ready-to-take oral tablet/capsule. Kits described herein can include instructions such as package inserts. In one embodiment, the instructions are package inserts—one for each agent in the kit.

Further provided are kits for carrying out the methods detailed herein, which comprise a combination therapy described herein and instructions for use in the treatment of breast cancer as described herein.

In one embodiment, the combination therapies described herein can be used for treating estrogen receptor-positive (ER+), human epidermal growth factor receptor 2− negative (HER2−) breast cancer. In another embodiment, the combination therapies described herein can be used for treating ER+, HER2− locally advanced breast cancer (IaBC) or ER+, HER2− metastatic breast cancer (mBC). In one such embodiment, the combination therapies described herein can be used for treating ER+, HER2− IaBC. In one such embodiment, the combination therapies described herein can be used for treating ER+, HER2− mBC.

Methods of Treating

Provided herein are methods of treating ER+, HER2− IaBC or mBC in a patient having such a cancer. In one aspect provided herein is a method (I1) of treating IaBC or mBC as described herein in a patient having such a cancer, where the method comprises administering to the patient a combination therapy comprising GDC-9545 or a pharmaceutically acceptable salt thereof and ipatasertib. In one embodiment of the method (I1) provided herein, the method is used for treating IaBC. In another embodiment of the method (I1) provided herein, the method is used for treating mBC.

Further provided herein is a method (I2) treating IaBC or mBC as described herein in a patient having such a cancer, where the method comprises administering to the patient a combination therapy as described herein comprising a dosing regimen comprising: (i) administering GDC-9545 or a pharmaceutically acceptable salt thereof QD on days 1-28 of a first 28-day cycle; and (ii) administering ipatasertib QD on days 1-21 of the first 28-day cycle. In one embodiment of the method (I2) provided herein, the method is used for treating IaBC. In another embodiment of the method (I2) provided herein, the method is used for treating mBC.

In one embodiment of the method of I1 or I2, GDC-9545 or a pharmaceutically acceptable salt thereof is administered as a fixed dose QD administration. In one embodiment, the administration is oral (PO), where GDC-9545 or a pharmaceutically acceptable salt thereof is formulated as a tablet or capsule. In one embodiment, GDC-9545 or a pharmaceutically acceptable salt thereof is administered at an amount of about 1 mg-100 mg, 1 mg-50 mg, 1 mg-30 mg, 10 mg-100 mg, 10 mg-50 mg, or 10 mg-30 mg QD. In another embodiment, GDC-9545 or a pharmaceutically acceptable salt thereof is administered at an amount of about 1, 5, 10, 15, 20, 25, 30, 50, or 100 mg. In still another embodiment, GDC-9545 or a pharmaceutically acceptable salt thereof is administered at an amount of about 30, 50, or 100 mg. In still another embodiment, GDC-9545 or a pharmaceutically acceptable salt thereof is administered at an amount of about 30 mg.

In one embodiment of method I1 or I2 described herein, ipatasertib is administered at an amount of 400 mg. Such administration can be in a single dose (i.e. a single or multiple pills). In one embodiment, the dose of ipatasertib is reduced to 300 mg or 200 mg when a patient described herein experiences an adverse event associated with treatment with ipatasertib or where, for example, the dose of ipatasertib is otherwise not tolerated by the patient during treatment. Ipatasertib can be administered PO QD as described herein.

Still further provided herein is a method (I3) of treating IaBC or mBC in a patient having such a cancer where the method comprises administering to the patient a combination therapy described herein comprising a dosing regimen comprising: (i) administering 30 mg GDC-9545 or a pharmaceutically acceptable salt thereof QD on days 1-28 of a first 28-day cycle; and (ii) administering 400 mg ipatasertib QD on days 1-21 of the first 28-day cycle. In one such embodiment, the dosing regimen includes 2 or more cycles as described herein. In one embodiment of the method (I3) provided herein, the method is used for treating IaBC. In another embodiment of the method (I3) provided herein, the method is used for treating mBC.

In one embodiment of the methods I1, I2, and I3, the cancer is inoperable locally advanced (IaBC) or metastatic ER+ breast cancer (mBC).

In one embodiment of the methods I1, I2, and I3, the combination of GDC-9545 or a pharmaceutically acceptable salt thereof and ipatasertib does not require co-administration (treatment) with gonadotropin releasing hormone (GnRH) agonist.

In one embodiment of the methods I1, I2, and I3, the administered amount of ipatasertib can be reduced. In one such embodiment, the dose of ipatasertib is reduced by 100 mg in a maximum of 2 total reductions (i.e. a reduction to 300 mg QD or to 200 mg QD). In one embodiment of the methods I1, I2, and I3, administration of one agent in the combination therapy (GDC-9454 or a pharmaceutically acceptable salt thereof or ipatasertib) can be interrupted by a maximum of 28 days. In one embodiment of the methods I1, I2, and I3, the dose of GDC-9545 is not reduced.

The methods I1, I2, and I3 of treating breast cancer as provided herein can include administration of a combination therapy described herein as part of a dosing regimen. In one embodiment, the dosing regimen comprises one or more cycles. In another embodiment, the dosing regimen comprises at least 2 cycles. In another aspect provided herein is the dosing regimen comprises 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, or 72 cycles. In still another embodiment, dosing regimen comprises about 2-72, 2-66, 2-60, 2-54, 2-48, 2-42, 2-36, 2-2-24, 2-18, or 2-12 cycles. In one embodiment, the dosing regimen includes administration of a combination therapy as described herein in any number of cycles until the desired response (e.g. OR, PFS, OS, ORR, DOR, CBR) reaches a desired outcome (e.g. increase in OR, PFS, OS, ORR, DOR, CBR compared to a control described herein). In another embodiment, the dosing regimen includes administration of a combination therapy as described herein in any number of cycles until toxicity develops or the patient otherwise experiences one or more adverse events (AEs) that prevents further administration. In still another embodiment, the dosing regimen includes administration of a combination therapy as described herein in any number of cycles until disease progression.

In one embodiment of the methods described herein, the patient is a postmenopausal woman.

In another embodiment of the methods described herein, the patient is a premenopausal or perimenopausal (i.e., not postmenopausal) woman. In one such embodiment, the patient is treated with LHRH agonist in combination with a combination therapy described herein. The LHRH agonist therapy may be initiated 28 days prior to Day 1 of Cycle 1. In one embodiment, the LHRH agonist is administered on Day 1 of each cycle.

In another embodiment of the methods described herein, the patient is a man. In one such embodiment, the patient is treated with a LHRH agonist in combination with a combination therapy described herein.

In one embodiment of the methods described herein, a patient described herein has been tested for the presence of estrogen receptor, prostaglandin receptor, or Ki67. In one embodiment of the methods described herein, a patient described herein has a documented ER-positive tumor according to American Society of Clinical Oncology/College of American Pathologists guidelines. In one such embodiment, a patient described herein has a documented HER2-negative tumor.

In one embodiment of the methods described herein, a patient described herein is treatment naive. In one such embodiment, a patient described herein has not received prior chemotherapy before administration of a combination therapy described herein. In another embodiment of the methods described herein, a patient described herein has not been previously treated with an aromatase inhibitor or a CDK4/6 inhibitor (e.g. palbociclib, abemaciclib, or ribociclib) or a combination thereof. In one such embodiment, the aromatase inhibitor is anastrozole, exemestane, or letrozole. In one embodiment, a patient described herein has not been previously treated with either letrozole or palbociclib or a combination thereof. In still another embodiment of the methods described herein, a patient described herein has not been previously treated with a SERD (e.g. fulvestrant) or with tamoxifen. In another embodiment of the methods described herein, a patient has not been previously treated with an AKT inhibitor.

In one embodiment of the methods described herein, a patient has been treated with one or more cancer therapies before administration of a combination therapy described herein. In another embodiment, a patient described herein has been previously treated with a PI3K inhibitor or a mTOR inhibitor prior to administration of the combination therapy. In another embodiment, a patient described herein has been previously treated with fulvestrant.

In one embodiment of the methods described herein, a patient has breast cancer described herein that is resistant to one or more cancer therapies. In one embodiment of the methods described herein, resistance to cancer therapy includes recurrence of cancer or refractory cancer. Recurrence may refer to the reappearance of cancer, in the original site or a new site, after treatment. In one embodiment of the methods described herein, resistance to a cancer therapy includes progression of the cancer during treatment with the anti-cancer therapy. In some embodiments of the methods described herein, resistance to a cancer therapy includes cancer that does not response to treatment. The cancer may be resistant at the beginning of treatment or it may become resistant during treatment. In some embodiments of the methods described herein, the cancer is at early stage or at late stage.

Systemic chemotherapy is considered as one standard of care (SOC) for patients with mBC, although no standard regimen or sequence exists. In one embodiment of the methods described herein, a patient described herein has been previously treated with one or more of the therapies selected from the group consisting of anastrozole, letrozole, exemestane, everolimus, palbociclib and letrozole, fulvestrant, trastuzumab and pertuzumab, or a combination thereof prior to administration of a combination therapy described herein.

In one embodiment of the methods described herein, a patient described herein can have IaBC or mBC as described herein that is resistant to one or more of the single agent therapies selected from the group consisting of anastrozole, letrozole, exemestane, everolimus, palbociclib and letrozole, fulvestrant, trastuzumab and pertuzumab, or a combination thereof.

In one embodiment of the methods described herein, a patient described herein may have undergone surgical treatment such as, for example, surgery that is breast-conserving (i.e., a lumpectomy, which focuses on removing the primary tumor with a margin), or more extensive (i.e., mastectomy, which aims for complete removal of all of the breast tissue) prior to administration of a combination therapy described herein. In another embodiment of the methods described herein, a patient described herein may undergo surgical treatment following treatment with a combination therapy described herein.

Radiation therapy is also administered post-surgery to the breast/chest wall and/or regional lymph nodes, with the goal of killing microscopic cancer cells left post-surgery. In the case of a breast conserving surgery, radiation is administered to the remaining breast tissue and sometimes to the regional lymph nodes (including axillary lymph nodes). In the case of a mastectomy, radiation may still be administered if factors that predict higher risk of local recurrence are present. In some embodiments of the methods provided herein a patient described herein may have received radiation therapy prior to administration of a combination therapy described herein. In other embodiments of the methods provided herein a patient described herein may have receive radiation therapy following administration of a combination therapy described herein.

In some embodiments of the methods described herein, a patient described herein does not have a history of other malignancy within 5 years prior to administration of a combination therapy described herein. In some embodiments of the methods described herein, a patient described herein does not have active inflammatory bowel disease, chronic diarrhea, short bowel syndrome, or major upper gastrointestinal surgery including gastric resection. In some embodiments of the methods described herein, a patient described herein does not have cardiac disease or cardiac dysfunction.

In one embodiment of the methods described herein, treatment with a combination therapy according to the methods provided herein increases a patient's OS comparable to a control (e.g. non-treatment, standard of care (SOC) treatment, or treatment with one agent described herein (e.g. GDC-9545 or ipatasertib) alone). In one embodiment of the methods described herein, treatment with a combination therapy according to the methods provided herein increases a patient's OS comparable to a control (e.g. non-treatment, standard of care (SOC) treatment, treatment with one agent described herein (e.g. GDC-9545 or ipatasertib) alone) by 1, 2, 3, 4, 5, 6, 7, 8, 9, 10, 11, 12, 14, 16, 18, 20, 24 or more months comparable to the control.

In one embodiment of the methods described herein, treatment with a combination therapy according to the methods provided herein increases the patient's amount of ORR. In one such embodiment, treatment with a combination therapy according to the methods provided herein results in more patients having a complete response (CR) or partial response (PR) than a control. In another embodiment of the methods described herein, the TTP is increased in a patient following treatment with a combination therapy according to the methods provided herein. In still another embodiment of the methods described herein, duration of response to the combination therapy is increased compared to a control (e.g. non-treatment, standard of care (SOC) treatment, treatment with one agent described herein (e.g. GDC-9545 or ipatasertib) alone). In one such embodiment, the duration of response is increased by at least 1-3, 2-6, 3-8, 4-10, 5-12, 6-15, 8-20, or 1-24 months. In still another embodiment of the methods described herein, a patient described herein has increased clinical benefit rate compared to a control (e.g. non-treatment, standard of care (SOC) treatment, treatment with GDC-9545 alone, or treatment with ipatasertib alone). In still another embodiment of the methods described herein, a patient has increased progression-free survival compared to a control (e.g. non-treatment, standard of care (SOC) treatment, treatment with GDC-9545 alone, or treatment with ipatasertib alone).

In one embodiment of the methods provided herein a patient is diagnosed having a CR following treatment with a combination therapy according to the methods provided herein. In one embodiment of the methods provided herein a patient is diagnosed having a PR following treatment with a combination therapy according to the methods provided herein. In one embodiment of the methods provided herein a patient is diagnosed having SD following treatment with a combination therapy according to the methods provided herein.

Further provided herein is the use (U1) of a combination therapy described herein comprising GDC-9545 or a pharmaceutically acceptable salt thereof and ipatasertib or a pharmaceutically acceptable salt thereof for the treatment of IaBC or mBC as described herein. In one embodiment, is a use (U2) of a combination therapy described herein comprising GDC-9545 or a pharmaceutically acceptable salt thereof and ipatasertib or a pharmaceutically acceptable salt thereof for the treatment of IaBC or mBC as described herein. In one embodiment, is a use (U3) of a combination therapy described herein comprising GDC-9545 or a pharmaceutically acceptable salt thereof and ipatasertib or a pharmaceutically acceptable salt thereof herein for the treatment of IaBC or mBC as described herein.

Further provided herein is the use (IU1) of a combination therapy described herein comprising GDC-9545 or a pharmaceutically acceptable salt thereof and ipatasertib as described herein for the treatment of mBC as described herein. Still further provided herein is the use (IU2) of a combination therapy described herein comprising GDC-9545 or a pharmaceutically acceptable salt thereof and ipatasertib as described herein for the treatment of IaBC as described herein.

Further provided herein is the use (IU3) of a combination therapy described herein comprising GDC-9545 or a pharmaceutically acceptable salt thereof and ipatasertib as described herein for the treatment of IaBC or mBC as described herein comprising a dosing regimen comprising: (i) administering GDC-9545 or a pharmaceutically acceptable salt thereof QD on days 1-28 of a first 28-day cycle; and (ii) administering ipatasertib QD on days 1-21 of the first 28-day cycle. In one embodiment of the use (IU3) provided herein, the combination therapy is for the treatment of IaBC. In another embodiment of the use (IU3) provided herein, the combination therapy is for the treatment of mBC.

Further provided herein is the use (IU4) of a combination therapy described herein comprising GDC-9545 or a pharmaceutically acceptable salt thereof and ipatasertib as described herein for the treatment of IaBC or mBC as described herein comprising a dosing regimen comprising: (i) administering 30 mg GDC-9545 or a pharmaceutically acceptable salt thereof QD on days 1-28 of a first 28-day cycle; and (ii) administering 400 mg ipatasertib QD on days 1-21 of the first 28-day cycle. In one such embodiment, the dosing regimen includes 2 or more cycles as described herein. In one embodiment of the use (IU4) provided herein, the combination therapy is for the treatment of IaBC. In another embodiment of the use (IU4) provided herein, the combination therapy is for the treatment of mBC.

Further provided herein is the use (IM1) of a combination therapy described herein comprising GDC-9545 or a pharmaceutically acceptable salt thereof and ipatasertib for the manufacture of a medicament for the treatment of IaBC or mBC as described herein. Still further provided herein is the use (IM2) of a combination therapy described herein comprising GDC-9545 or a pharmaceutically acceptable salt thereof and ipatasertib for the manufacture of a medicament for the treatment of mBC as described herein. Further provided herein is the use (IM3) of a combination therapy described herein comprising GDC-9545 or a pharmaceutically acceptable salt thereof and ipatasertib for the manufacture of a medicament for the treatment of IaBC as described herein.

Further provided herein is the use (IM4) of a combination therapy described herein comprising GDC-9545 or a pharmaceutically acceptable salt thereof and ipatasertib for the manufacture of a medicament for the treatment of IaBC or mBC as described herein comprising a dosing regimen comprising: (i) administering GDC-9545 or a pharmaceutically acceptable salt thereof QD on days 1-28 of a first 28-day cycle; and (ii) administering ipatasertib QD on days 1-21 of the first 28-day cycle. In one embodiment of the use (IM4) provided herein, the combination therapy is for the treatment of IaBC. In another embodiment of the use (IM4) provided herein, the combination therapy is for the treatment of mBC.

Further provided herein is the use (IM5) of a combination therapy described herein comprising GDC-9545 or a pharmaceutically acceptable salt thereof and ipatasertib for the manufacture of a medicament for the treatment of IaBC or mBC as described herein comprising a dosing regimen comprising: (i) administering 30 mg GDC-9545 or a pharmaceutically acceptable salt thereof QD on days 1-28 of a first 28-day cycle; and (ii) administering 400 mg ipatasertib on days 1-21 of the first 28-day cycle. In one such embodiment, the dosing regimen includes 2 or more cycles as described herein. In one embodiment of the use (IM5) provided herein, the combination therapy is for the treatment of IaBC. In another embodiment of the use (IM5) provided herein, the combination therapy is for the treatment of mBC.

AIso provided herein are methods of inhibiting tumor growth or producing tumor regression in a patient described herein by administering a combination therapy described herein. In one embodiment provided herein is a method of inhibiting tumor growth in a patient having IaBC described herein by administering a combination therapy comprising administering GDC-9545 or a pharmaceutically acceptable salt thereof and ipatasertib in one or more 28-day cycles as described herein. In one embodiment provided herein is a method of inhibiting tumor growth in a patient having mBC described herein by administering a combination therapy comprising administering GDC-9545 or a pharmaceutically acceptable salt thereof and ipatasertib in one or more 28-day cycles as described herein.

In one embodiment provided herein is a method of producing or improving tumor regression in a patient having mBC described herein by administering a combination therapy comprising administering GDC-9545 or a pharmaceutically acceptable salt thereof and ipatasertib in one or more 28-day cycles as described herein. In one embodiment provided herein is a method of producing or improving tumor regression in a patient having IaBC described herein by administering a combination therapy comprising administering GDC-9545 or a pharmaceutically acceptable salt thereof and ipatasertib in one or more 28-day cycles as described herein.

The development of combination treatments poses challenges including, for example, the selection of agents for combination therapy that may lead to improved efficacy while maintaining acceptable toxicity. One particular challenge is the need to distinguish the incremental toxicity of the combination. In one embodiment of the methods described herein the combination therapy described herein (e.g. GDC-9545 or a pharmaceutically acceptable salt thereof and ipatasertib) is administered in a dosing regimen comprising a staggered dosing schedule. In one such embodiment, the patient has a reduced number or grade of adverse events (AEs) comparable to a control (e.g. SOC therapy, treatment with one agent described herein (e.g. GDC-9545 or ipatasertib) alone).

In one embodiment of the methods described herein, the dosing regimen reduces the number or frequency of grade 2 or grade 3 or higher grade adverse event comparable to administration of either GDC-9545 or ipatasertib alone. In one such embodiment, the dosing regimen eliminates the number or frequency of grade 3 or higher AEs. In one embodiment, the dosing regimen reduces the grade of bradycardia or QT prolongation.

In another embodiment of the methods described herein the dosing reduces the number or frequency of grade 2 or grade 3 or higher grade adverse event comparable to administration of either agent alone.

It is generally understood that the when an adverse event occurs, four options exist: (1) continue treatment as-is with optional concomitant therapy; (2) adjust the dose of one or more agents in the dosing regiment; (3) suspend administration of one or more agents in the dosing regimen; or (4) discontinue administration of one or more agents in the dosing regimen. In one embodiment, GDC-9545 is not adjusted.

In one embodiment of the methods described herein, a patient described herein experiences one or more adverse events comprising rash, bradycardia, hyperglycemia, diarrhea, nausea, or pruritus. In one such embodiment, a patient described herein has the same level or reduced level/severity of one or more of such AEs. In another embodiment, a patient described herein has a reduced severity of one or more of such AEs. In one embodiment, a patient described herein has a reduced severity of hyperglycemia, diarrhea, or bradycardia compared to a control. In one such embodiment, the control is (i) either agent alone or (ii) SOC therapy.

In one embodiment, a patient described herein has the same level or reduced level of hyperglycemia following administration of the combination therapy compared to the control. In one such embodiment, the control is ipatasertib alone. In still another embodiment, a patient described herein has the same level or reduced level of bradycardia following administration of the combination therapy compared to GDC-9545 alone.

In one embodiment, the adverse event(s) experienced by a patient described herein undergoing treatment with a combination therapy described herein are comparably reduced as described herein.

In one embodiment of the methods described herein, a patient described herein experiences an adverse event comprising diarrhea. In one embodiment of the methods described herein, a patient described herein experiences an adverse event comprising hyperglycemia. In one embodiment of the methods described herein, a patient described herein experiences an adverse event comprising bradycardia. In some embodiments, where a patient experiences one or more AEs selected from the group consisting of hyperglycemia, diarrhea, and bradycardia from treatment with a combination therapy described herein, the severity is Grade 2 or less. In one embodiment, a patient described herein does not experience one or more AEs selected from the group consisting of hyperglycemia, diarrhea, and bradycardia from treatment with a combination therapy described herein, where the severity of the AE is higher than Grade 2.

Biomarkers

Breast cancer is a heterogeneous disease with many distinct subtypes as defined by molecular signatures and a diverse array of mutational profiles. Patients described herein can be tested for ER+ HER2− IaBC or mBC using diagnostic methods, or kits to inform treating or predict of responsiveness of a patient to the combination therapies described herein. In one embodiment, a patient can be tested by determining an ER pathway activity score such as those described in US Patent Application Publication 20200082944. In some embodiments, a patient sample is taken and tested to determine an ER pathway activity score. The score can be calculated using a 41-gene signature by subtracting an E2-repressed score (as determined from the average z-scored expression of genes comprising BAMBI, BCAS1, CCNG2, DDIT4, EGLN3, FAM171B, GRM4, IL1 R1, LIPH, NBEA, PNPLA7, PSCA, SEMA3E, SSPO, STON1, TGFB3, TP531NP1, and TP531NP2) from an E2-induced score (as determined from the average z-scored expression of genes set forth in AGR3, AMZ1, AREG, C5AR2, CELSR2, CT62, FKBP4, FMN1, GREB1, IGFBP4, NOS1AP, NXPH3, OLFM1, PGR, PPM1J, RAPGEFL1, RBM24, RERG, RET, SGK3, SLC9A3R1, TFF1, and ZNF703).

In one embodiment, the sample from the patient used for determining the ER pathway activity score is a tumor tissue sample, (e.g., a formalin-fixed paraffin-embedded (FFPE), a fresh frozen (FF), an archival, a fresh, or a frozen tumor tissue sample).

In some instances, a patient described herein is administered a combination therapy described herein where the measured ER pathway activity score is be between about −1.0 to about −0.2 (e.g., between about −0.9 to about −0.2, e.g., between about −0.8 to about −0.2, e.g., between about −0.7 to about −0.2, e.g., between about −0.6 to about −0.2, e.g., between about −0.5 to about −0.2, e.g., between about −0.4 to about −0.2, or e.g., between about −0.3 to about −0.2). In some instances, the ER activity score from the sample may be less than −1.0.

In some embodiments, samples of patients described herein can be assessed for additional biomarkers in an effort to identify factors that may correlate with the safety and efficacy of the study treatments.

In one embodiment of the methods described herein, NGS, whole genome sequencing (WGS), other methods, or a combination thereof is used for DNA obtained from blood samples and tumor tissue from patients described herein. Such samples may be analyzed to identify germline and somatic alterations that are predictive of response to study drug, are associated with progression to a more severe disease state, are associated with acquired resistance to study drug, or can increase the knowledge and understanding of disease biology.

In one embodiment, a patient can be tested for PIK3CA/AKT1/PTEN—alteration status. In one embodiment, a patient described herein can be tested for one or more of a phosphatase and tensin homolog (PTEN) mutation, PTEN loss (or loss of PTEN function), a phosphatidylinositol-4,5-bisphosphate 3-kinase catalytic subunit alpha (PIK3CA) mutation, a protein kinase B alpha (AKT1) mutation, or a combination thereof. In one such embodiment, a patient described herein has a breast cancer comprising a PIK3CA mutation selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M10431/T/V, G1049A/C/D/R/S, E453A/D/G/K/Q/V, K111N/R/E, G106A/D/R/S/V, G118D, and R88Q. In one embodiment, the patient has breast cancer expressing a PIK3CA mutant comprising a mutation corresponding to positions selected from the group consisting of E542K, E545K, Q546R, H1047L and H1047R. In one embodiment, the patient has mutant PIK3CA comprising a mutation corresponding to positions containing one mutation selected from the group consisting of E542K, E545K, Q546R, H1047L and H1047R, and a second mutation selected from the group consisting of E453Q/K, E726K and M1043L/I. In one embodiment, the patient has breast cancer expressing a PIK3CA mutant comprising a mutation corresponding to positions selected from the group consisting of E542K+E453Q/K, E542K +E726K, E542K+M1043L/I; E545K+E453Q/K, E545K+E726K, E545K+M1043L/I; H1047R+E453Q/K, and H1047R+E726K. In one embodiment, PIK3CA-mutant tumor status is assessed by either central testing of blood or local testing of blood or tumor tissue.

In another such embodiment, samples of patients described herein can be assessed for additional biomarkers in an effort to identify factors that may correlate with the safety and efficacy of the study treatments. In one embodiment, a patient described herein has a tumor comprising loss of PTEN as characterized by, for example, IHC or NGS testing. In another embodiment, a patient described herein has a tumor comprising one or more amino acid mutations of PTEN. In still another embodiment, a patient described herein has a tumor comprising one or more amino acid mutations of AKT corresponding to positions E17, L52, or Q79.

Circulating tumor DNA (ctDNA) can be detected in the blood of cancer patients with epithelial cancers and may have diagnostic and therapeutic significance. For example, the mutational status of tumor cells may be obtained through the isolation of ctDNA (Maheswaran S, et al. N Engl J Med 2008; 359:366-77), and ctDNA has been used to monitor treatment effectiveness in melanoma (Shinozaki M, et al. Clin Cancer Res 2007; 13:2068-74). Blood samples from patients described herein can be collected at screening, at time of first tumor assessment, and/or at the study completion/early termination visit.

In one embodiment, patients are tested for the presence, level, or amount of a compound having structure:

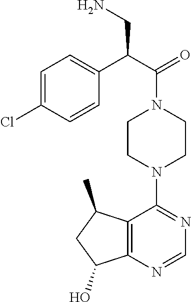 [SMILES: C[C@@H]1C[C@@H](O)c2ncnc(N3CCN(C(=O)[C@H](CN)c4ccc(Cl)cc4)CC3)c21]

having the chemical name, (S)-3-amino-2-(4-chlorophenyl)-1-(4-((5R,7R)-7-hydroxy-5-methyl-6,7-dihydro-5H-cyclopenta[d]pyrimidin-4-Apiperazin-1-yl)propan-1-one, which is a metabolite of ipatasertib.

### EMBODIMENTS

Provided below are exemplary embodiments of the invention.

Embodiment No 1. A combination therapy comprising GDC-9545 or a pharmaceutically acceptable salt thereof administered QD on days 1-28 of a first 28-day cycle and ipatasertib or a pharmaceutically acceptable salt thereof administered QD on days 1-21 of the first 28-day cycle.

Embodiment No 2. The combination therapy of embodiment 1, wherein ipatasertib or a pharmaceutically acceptable salt thereof is administered at a dose of 400 mg.

Embodiment No 3. The combination therapy of embodiment 1 or embodiment 2, wherein GDC-9545 or a pharmaceutically acceptable salt thereof is administered at an amount of about 10 mg to about 100 mg.

Embodiment No 4. The combination therapy of any one of embodiments 1-3, wherein GDC-9545 or a pharmaceutically acceptable salt thereof is administered at an amount of about 10, 30, 50, or 100 mg.

Embodiment No 5. The combination therapy of any one of embodiments 1-4, wherein GDC-9545 or a pharmaceutically acceptable salt thereof is administered at an amount of 30 mg.

Embodiment No 6. The combination therapy of any one of embodiments 1-5, wherein the dosing regimen comprises 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, or 72 cycles.

Embodiment No 7. The combination therapy of any one of embodiments 1-5, wherein the dosing regimen comprises about 2-72, 2-66, 2-60, 2-54, 2-48, 2-42, 2-36, 2-30, 2-24, 2-18, or 2-12 cycles.

Embodiment No 8. A method of treating estrogen receptor-positive and HER2-negative locally advanced breast cancer (IaBC) or metastatic breast cancer (mBC) in a patient having estrogen receptor-positive and HER2-negative IaBC or mBC, the method comprising administering to the patient a combination therapy comprising GDC-9545 or a pharmaceutically acceptable salt thereof and ipatasertib or a pharmaceutically acceptable salt thereof, wherein said combination therapy is administered over a 28-day cycle.

Embodiment No 9. The method of embodiment 8, wherein the combination therapy further comprises a dosing regimen comprising:
(i) administering GDC-9545 or a pharmaceutically acceptable salt thereof QD on days 1-28 of a first 28-day cycle; and
(ii) administering ipatasertib or a pharmaceutically acceptable salt thereof QD on days 1-21 of the first 28-day cycle.

Embodiment No 10. The method of embodiment 8 or embodiment 9, wherein ipatasertib or a pharmaceutically acceptable salt thereof is administered at a dose of 400 mg.

Embodiment No 11. The method of any one of embodiments 8-10, wherein GDC-9545 or a pharmaceutically acceptable salt thereof is administered at an amount of about mg to about 100 mg.

Embodiment No 12. The method of any one of embodiments 8-11, wherein GDC-9545 or a pharmaceutically acceptable salt thereof is administered at an amount of about 30, 50, or 100 mg.

Embodiment No 13. The method of any one of embodiments 8-11, wherein GDC-9545 or a pharmaceutically acceptable salt thereof is administered at an amount of 30 mg.

Embodiment No 14. The method of any one of embodiments 8-13, wherein the dosing regimen comprises 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, or 72 cycles.

Embodiment No 15. The method of any one of embodiments 8-13, wherein the dosing regimen comprises about 2-72, 2-66, 2-60, 2-54, 2-48, 2-42, 2-36, 2-30, 2-24, 2-18, or 2-12 cycles.

Embodiment No 16. The method of any one of embodiments 8-15, wherein the patient is premenopausal.

Embodiment No 17. The method of any one of embodiments 8-16, wherein the patient is male.

Embodiment No 18. The method of any one of embodiments 8-17, wherein the patient is tested for the presence of a mutation of one or more of estrogen receptor, prostaglandin receptor, or Ki67.

Embodiment No 19. The method of any one of embodiments 8-18, wherein the patient has a tumor comprising loss of phosphatase and tensin homolog (PTEN).

Embodiment No 20. The method of any one of embodiments 8-18, wherein the patient has a tumor comprising mutation of phosphatase and tensin homolog (PTEN).

Embodiment No 21. The method of any one of embodiments 8-19, wherein the patient has a tumor comprising mutation of AKT1 corresponding to position E17, L52, or Q79.

Embodiment No 22. The method of any one of embodiments 8-21, wherein the patient has reduced adverse events (AEs) comparable to a control.

Embodiment No 23. The method of embodiment 22, wherein the patient has reduced severity of one or more AEs selected from the group consisting of hyperglycemia, bradycardia, diarrhea, nausea, or pruritus compared to the control.

Embodiment No 24. The method of embodiment 22, wherein the patient has the same level or reduced level of bradycardia following administration of the combination therapy compared to the control.

Embodiment No 25. The method of any one of embodiments 8-24, wherein the patient has an increased overall survival (OS) comparable to a control.

Embodiment No 26. The method of embodiment 25, wherein the patient has an increased overall survival (OS) of 1, 2, 3, 4, 5, 6, 7, 8, 9, 10, 11, 12, 14, 16, 18, 20, 24 or more months comparable to a control.

Embodiment No 27. The method of any one of embodiments 8-26, wherein duration of response to the combination therapy is increased compared to a control.

Embodiment No 28. The method of embodiment 27, wherein the duration of response is increased by at least 1-3, 2-6, 3-8, 4-10, 5-12, 6-15, 8-20, or 1-24 months.

Embodiment No 29. The method of any one of embodiments 8-28, wherein a patient has increased progression-free survival compared to a control.

Embodiment No 30. The method of embodiment 29, wherein the increase is at least 2, 4, 6, 8, 10, 12, 14, 16, 18, 20, 22, 24, 26, 28, 30, 36, 42, 48, 50, 54, 60, 66, or 72 months.

Embodiment No 31. The method any one of embodiments 22-30, wherein the control is GDC-9545 or a pharmaceutically acceptable salt thereof administered alone ipatasertib or a pharmaceutically acceptable salt thereof administered alone.

Embodiment No 32. The method of any one of embodiments 8-31, wherein the patient has not received prior chemotherapy before administration of the combination therapy.

Embodiment No 33. The method of any one of embodiments 8-31, wherein the patient has been previously treated with tamoxifen.

Embodiment No 34. The method of any one of embodiments 8-31, wherein the patient has been previously treated with a PI3K inhibitor or a mTOR inhibitor prior to administration of the combination therapy.

Embodiment No 35. The method of any one of embodiments 8-31, wherein the patient has not been previously treated with an aromatase inhibitor or a CDK4/6 inhibitor or a combination thereof.

Embodiment No 36. A kit comprising the combination therapy of embodiment 1 and instructions for use.

Embodiment No 37. The kit of embodiment 36, wherein GDC-9545 or a pharmaceutically acceptable salt thereof and ipatasertib or a pharmaceutically acceptable salt thereof are co-formulated.

Embodiment No 38. Use of a combination therapy comprising GDC-9545 or a pharmaceutically acceptable salt thereof and ipatasertib or a pharmaceutically acceptable salt thereof for the treatment of IaBC or mBC.

Embodiment No 39. Use of a combination therapy comprising GDC-9545 or a pharmaceutically acceptable salt thereof and ipatasertib or a pharmaceutically acceptable salt thereof for the manufacture of a medicament for the treatment of IaBC or mBC.

Embodiment No 40. The use of embodiment 38 or 39, wherein the combination therapy comprises a dosing regimen comprising: (i) administering 30 mg GDC-9545 or a pharmaceutically acceptable salt thereof QD on days 1-28 of a first 28-day cycle; and (ii) administering ipatasertib or a pharmaceutically acceptable salt thereof on days 1-21 of the first 28-day cycle.

Embodiment No 41. The use of any one of embodiments 38-40, wherein the combination therapy is for the treatment of IaBC.

Embodiment No 42. The use of any one of embodiments 38-40, wherein the combination therapy is for the treatment of mBC.

Embodiment No 43. A method of inhibiting tumor growth in a patient having IaBC or mBC, the method comprising administering a combination therapy comprising GDC-9545 or a pharmaceutically acceptable salt thereof and ipatasertib or a pharmaceutically acceptable salt thereof in one or more 28-day cycles.

Embodiment No 44. A method of producing or improving tumor regression in a patient having IaBC or mBC, the method comprising administering a combination therapy comprising GDC-9545 or a pharmaceutically acceptable salt thereof and ipatasertib or a pharmaceutically acceptable salt thereof in one or more 28-day cycles.

The following Examples are presented by way of illustration, not limitation.

### EXAMPLES

The role of estrogen in breast cancer etiology and disease progression is well established (Colditz et al. N Engl J Med 1995; 332:1589-93). Modulation of estrogen activity and/or synthesis is one therapeutic approach in patients with ER+ breast cancer.

Despite the effectiveness of available therapies for patients with ER+, locally advanced or metastatic disease including endocrine therapy (ET) and combinations of endocrine and targeted therapy, many patients ultimately relapse or develop resistance to these agents and therefore require further treatment for optimal disease control. However, growth and survival of the majority of tumors are thought to remain dependent on ER signaling, despite becoming refractory to AIs or tamoxifen. Patients with ER+ breast cancer can still respond to second- or third-line ET after progression on prior therapy (Di Leo et al. J Clin Oncol. 2010; 28:4594-600; Baselga et al. N Engl J Med. 2012; 366:520-9). Without being bound by any particular theory, there is evidence that in the endocrine-resistant state, the ER can signal in a ligand-independent manner (Miller et al. J Clin Invest 2010; 120:2406-13; Van Tine et al. Cancer Discov 2011; 1:287-8). An agent (or combination of agents) capable of targeting both ligand-dependent and ligand-independent ER signaling has the potential to improve treatment outcomes in patients with ER+ breast cancer.

ESR1 mutations appear to be a major mechanism of acquired resistance to AIs and are associated with poorer outcomes (Schiavon et al. Sci Transl Med 2015; 7:313ra182; Chandarlapaty et al. JAMA Oncol 2016; 2:1310-15; Fribbens et al. J Clin Oncol 2016; 34:2961-8). The prevalence of ESR1 mutation appears to range from about 25%-40% after AI exposure but only in 2%-3% of ET-naive patients (Chandarlapaty et al. 2016). This illustrates that ESR1 becomes one important oncogenic driver under AI-selection pressure. Studies have identified mutations in ESR1 encoding ER-α (primarily Y537S and D538G) affecting the ligand binding domain “LBD” of the ER-α (Segal and Dowsett Clin Cancer Res 2014; 20:1724-6). Studies using clinical samples and nonclinical models describe ER antagonists appear efficacious against ligand-independent, constitutively active ER—mutated receptors and may have therapeutic benefit for patients that were resistant to AIs (Li et al. Cell Rep. 2013; 4:1116-30; Merenbakh-Lamin et al. Cancer Res 2013; 73:6856-64; Robinson et al. Nat Genet 2013; 45:1466-51; Toy et al. Nat Genet 2013; 45:1439-45; Alluri et al. Breast Cancer Res 2014; 16:494; Segal and Dowsett Clin Cancer Res 2014; 20:1724-6; Jeselsohn et al. Nat Rev Clin Oncol 2015; 12:573-83; Niu et al. Onco Targets Ther. 2015; 8:3323-8; Schiavon et al. Sci Transl Med 2015; 7:313ra182; Chu et al. Clin Cancer Res 2016; 22:993-9).

Selective estrogen receptor degraders (SERDs) can block endocrine-dependent and endocrine-independent ER signaling and have been recognized to offer a therapeutic approach to ER+ metastatic breast cancer. Fulvestrant, a first-generation SERD, binds, blocks, and degrades the ER, leading to inhibition of estrogen signaling through the ER. Fulvestrant has also shown benefit over anastrozole in frontline patients, as demonstrated in one study (NCT01602380). However, bioavailability and delivery of fulvestrant hinder its effectiveness administration.

Nonclinical studies comparing drug exposure and in vitro potency of GDC-9545 versus fulvestrant demonstrated that human steady-state total drug exposure of GDC-9545 at 30 mg once a day (QD) is approximately 10-fold higher than the steady-state exposure of fulvestrant 500 mg intramuscular (IM) monthly. Furthermore, the lower plasma protein binding of GDC-9545 provides higher free concentration of GDC-9545 than fulvestrant. In in vitro cell and biochemical assays, GDC-9545 exhibited up to 10-times higher potency than fulvestrant both in wild-type and ESR1-mutant contexts. Fulvestrant, when dosed according to a clinically relevant dosing scheme, was less efficacious than GDC-9545 in the assessed xenograft models.

Akt is a central node of the PI3K/Akt/mammalian target of rapamycin (mTOR) signaling axis and represents a major downstream effector of receptor tyrosine kinases. Without being bound by any particular theory, the activation of the PI3K/Akt pathway results in essential cellular functions including cell survival, growth, and proliferation, which are properties that underlie human cancers. The PI3K/Akt pathway can be activated, for example, through loss of the tumor suppressor PTEN (Li et al. Science 1997; 275:1943-7), through activating mutations and/or amplifications in PIK3CA (Bachman et al. Cancer Biot Ther 2004; 3:772-5), or through activating mutations in AKT1 (Carpten et al. Nature 2007; 448:439-44); all these events are frequently observed in HR+ breast cancer.

Up to 70% of breast cancers can have some form of molecular aberration of the PI3K/Akt/mTOR pathway (Cancer Genome Atlas Network 2012). Among the breast cancer subtypes, HR+ breast cancer is associated with the highest prevalence of PI3K pathway activating mutations, making up about 50% of the total HR+ breast cancers (Curtis et al. Nature 2012; 486:346-52; Cancer Genome Atlas Network 2012; Wilson et al. NPJ Breast Cancer 2016; 2:16022). These abnormalities include PTEN alterations and AKT1 and/or PIK3CA mutations.

The PI3K/Akt/mTOR pathway, like other mitogenic pathways, such as the MAPK, NF-kB/IKK, and the ERs, can provide the interaction between cyclin D and CDK4/6 (Miller et al. J Clin Invest 2010; 120:2406-13). Herrera-Abreu and colleagues reported that chronic inhibition by CDK4/6i therapies was associated with increased AKT phosphorylation, which correlated with the sustained expression of cyclin E2 or CDK2, preventing the inhibition of Rb phosphorylation (Herrera-Abreu et al. Cancer Res 2016; 76: 2301-13). Furthermore, a study using serial biopsies from patients uncovered PTEN loss as a mechanism of acquired resistance of CDK4/6i therapies (Costa et al. Cancer Discov 2020; 10:72-8). In addition, a study using CDK4/6i—resistant breast cancer cell line showed that these cells remain responsive to PI3K/Akt/mTOR pathway inhibitors such as alpelisib and everolimus in cell growth analysis, suggesting PI3K/Akt/mTOR pathway inhibitors may serve as optimal treatment options for patients whose cancer had progressed following treatment with CDK4/6i therapies (lida et al. Breast Cancer 2020;10.1007/s12282-020-01090-3).

GDC-9545 is a potent, orally bioavailable ER-α antagonist and inducer of ER-α degradation that competes with estrogens for binding to the ER with low nanomolar potency; it is being developed for the treatment of patients with ER+ advanced or metastatic breast cancer. GDC-9545 has demonstrated robust nonclinical activity in ER+ breast cancer models of ESR1—wild type and ESR1-mutation—bearing disease. Furthermore, fulvestrant, an approved SERD molecule, when dosed according to a clinically relevant dosing scheme, was less efficacious than GDC-9545 in the assessed xenograft models).

GDC-9545 and ipatasertib likely show synergistic activity and each have preliminary efficacy data and manageable safety profiles. Treatment with GDC-9545 plus ipatasertib has promising therapeutic potential for patients ER+, and HER2-negative advanced breast cancer.

Patients will receive GDC-9545 at a dose of 30 mg PO QD during each 28 day cycle and ipatasertib at a dose of 400 mg PO QD on Days 1-21 of each 28 day cycle.

Patients administered GDC-9545 should be taken PO at approximately the same time each day starting with Day 1 of Cycle 1, and on Day 1 of each 28-day cycle thereafter. If a dose is not taken within 6 hours after the scheduled dosing, it will be considered missed. If a dose is missed or vomited, the patient should resume dosing with the next scheduled dose; missed or vomited doses will not be made up. Ipatasertib should be taken at approximately the same time each day, and no later than 4 hours after the scheduled time.

Patients administered GDC-9545 and ipatasertib are permitted to use the following concomitant therapies: a) Symptomatic anti-emetics, anti-diarrheal therapy, and other palliative and supportive care for disease-related symptoms; b) Pain medications administered per standard clinical practice; and/or c) Bone-sparing agents (e.g., bisphosphonates, denosumab) for the treatment of osteoporosis/osteopenia or for palliation of bone metastases, provided patient was on stable doses prior to Day 1 of Cycle 1.

Patients administered GDC-9545 and ipatasertib are not permitted to use the following concomitant therapies:
a. Investigational therapy (other than protocol-mandated study treatment) is within 28 days prior to first dose of GDC9545 and ipatasertib.
b. Any concomitant therapy intended for the treatment of cancer including, but not limited to, chemotherapy, immunotherapy, biologic therapy, radiotherapy, or herbal therapy is prohibited.
c. Hormone replacement therapy, topical estrogens (including any intra-vaginal preparations), megestrol acetate, and selective ER modulators (e.g., raloxifene).
d. Primary prophylactic use of hematopoietic growth factors (e.g., erythropoietins, granulocyte colony-stimulating factor, and granulocyte-macrophage colony-stimulating factor).
e. Radiotherapy for unequivocal progressive disease, with the exception of new brain metastases in the setting of systemic response as follows:
   Patients who have demonstrated control of their systemic disease (defined as having received clinical benefit [i.e., a PR, CR, or SD for 24 weeks]), but who have developed isolated brain metastases treatable with radiation.
   ET (i.e., GDC-9545) may be administered concomitantly with radiotherapy. f. Quinidine or other anti-arrhythmic agents

GDC-9545 may temporarily be suspended in patients experiencing toxicity considered to be related to study treatment. Ipatasertib may temporarily be suspended in patients experiencing toxicity considered to be related to study treatment. If either GDC-9545 or ipatasertib is discontinued, the other drug can be continued if the patient is likely to derive clinical benefit, as determined by the investigator.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed herein. The upper and lower limits of these small ranges which can independently be included in the smaller rangers is also encompassed herein, subject to any specifically excluded limit in the stated range. Where the stated range includes one or both of the limits, ranges excluding either or both of those included limits are also included herein.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Biology Assays

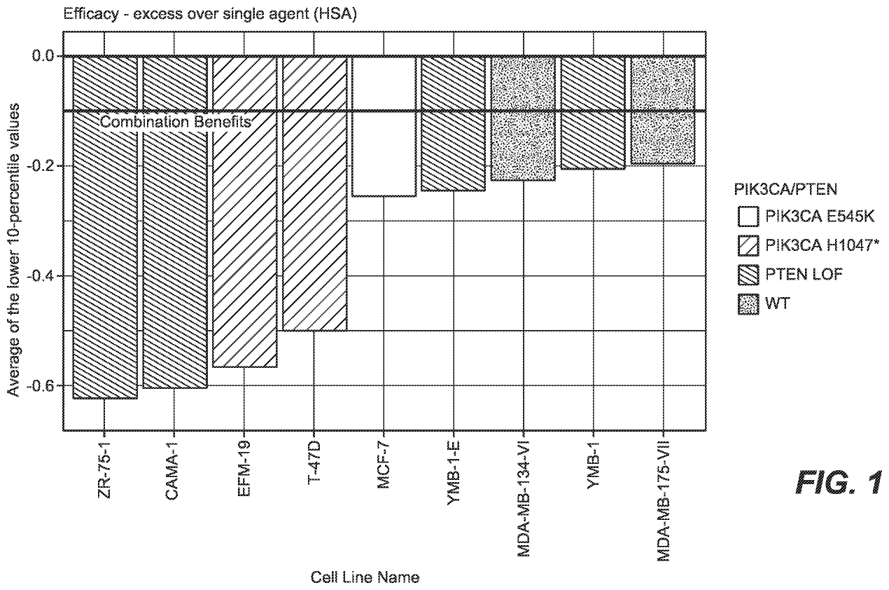

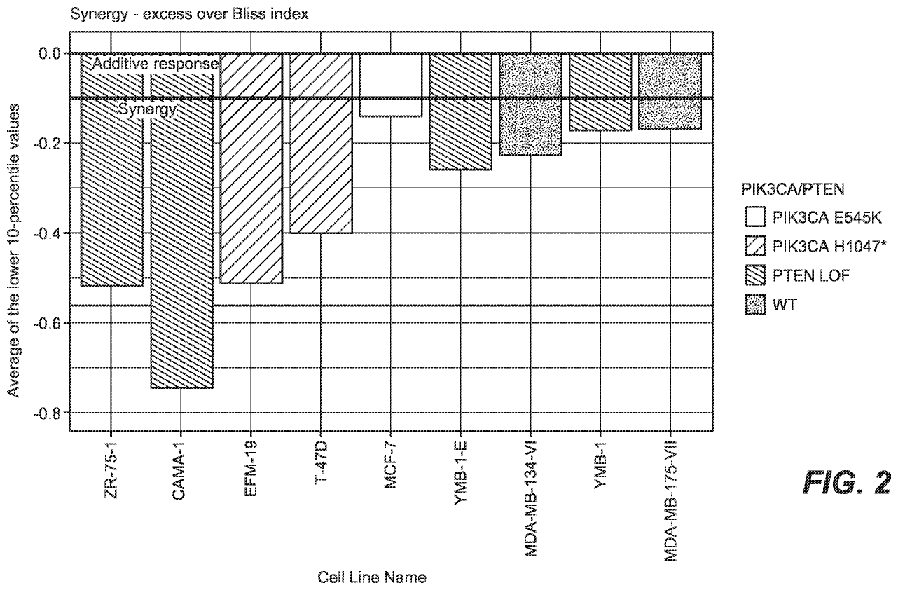

HR+ cell lines ZR-75-1, CAMA-1, EFM-19, T47D, MCF-7, YMB-1-E, MDA-MB-134-VI, YMB-1, and MDA-MB-175-VII were contacted with GDC-9545 (0-10 uM) and GDC-0068 (ipatasertib, 0-5 uM). Depicted cell lines in FIG. 1 and FIG. 2 were further characterized on their expression of PIK3CA mutations (E545K and H1047x) and loss of function of PTEN. Two cell lines (MDA-MB-134-VI and MDA-MB-175-VII) lack these mutations and are considered wildtype (WT).

Combination benefit and synergy were calculated according to the methods set forth in Hafner et al. (Nature Methods volume 13, pages521-527(2016)) and Hafner et al. (Nature Biotechnology volume 35, pages500-502(2017)).

What is claimed is:

1. A combination therapy comprising GDC-9545 or a pharmaceutically acceptable salt thereof administered QD on days 1-28 of a first 28-day cycle and ipatasertib or a pharmaceutically acceptable salt thereof administered QD on days 1-21 of the first 28-day cycle.

2. The combination therapy of claim 1, wherein ipatasertib or a pharmaceutically acceptable salt thereof is administered at a dose of 400 mg.

3. The combination therapy of claim 1 or claim 2, wherein GDC-9545 or a pharmaceutically acceptable salt thereof is administered at an amount of about 10 mg to about 100 mg.

4. The combination therapy of any one of claims 1-3, wherein GDC-9545 or a pharmaceutically acceptable salt thereof is administered at an amount of about 10, 30, 50, or 100 mg.

5. The combination therapy of any one of claims 1-4, wherein GDC-9545 or a pharmaceutically acceptable salt thereof is administered at an amount of 30 mg.

6. The combination therapy of any one of claims 1-5, wherein the dosing regimen comprises 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, or 72 cycles.

7. The combination therapy of any one of claims 1-5, wherein the dosing regimen comprises about 2-72, 2-66, 2-60, 2-54, 2-48, 2-42, 2-36, 2-30, 2-24, 2-18, or 2-12 cycles.

8. A method of treating estrogen receptor-positive and HER2-negative locally advanced breast cancer (IaBC) or metastatic breast cancer (mBC) in a patient having estrogen receptor-positive and HER2-negative IaBC or mBC, the method comprising administering to the patient a combination therapy comprising GDC-9545 or a pharmaceutically acceptable salt thereof and ipatasertib or a pharmaceutically acceptable salt thereof, wherein said combination therapy is administered over a 28-day cycle.

9. The method of claim 8, wherein the combination therapy further comprises a dosing regimen comprising:
(i) administering GDC-9545 or a pharmaceutically acceptable salt thereof QD on days 1-28 of a first 28-day cycle; and
(ii) administering ipatasertib or a pharmaceutically acceptable salt thereof QD on days 1-21 of the first 28-day cycle.

10. The method of claim 8 or claim 9, wherein ipatasertib or a pharmaceutically acceptable salt thereof is administered at a dose of 400 mg.

11. The method of any one of claims 8-10, wherein GDC-9545 or a pharmaceutically acceptable salt thereof is administered at an amount of about 10 mg to about 100 mg.

12. The method of any one of claims 8-11, wherein GDC-9545 or a pharmaceutically acceptable salt thereof is administered at an amount of about 10, 30, 50, or 100 mg.

13. The method of any one of claims 8-11, wherein GDC-9545 or a pharmaceutically acceptable salt thereof is administered at an amount of 30 mg.

14. The method of any one of claims 8-13, wherein the dosing regimen comprises 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, or 72 cycles.

15. The method of any one of claims 8-13, wherein the dosing regimen comprises about 2-72, 2-66, 2-60, 2-54, 2-48, 2-42, 2-36, 2-30, 2-24, 2-18, or 2-12 cycles.

16. The method of any one of claims 8-15, wherein the patient is premenopausal.

17. The method of any one of claims 8-16, wherein the patient is male.

18. The method of any one of claims 8-17, wherein the patient is tested for the presence of a mutation of one or more of estrogen receptor, prostaglandin receptor, or Ki67.

19. The method of any one of claims 8-18, wherein the patient has a tumor comprising loss of phosphatase and tensin homolog (PTEN).

20. The method of any one of claims 8-18, wherein the patient has a tumor comprising mutation of phosphatase and tensin homolog (PTEN).

21. The method of any one of claims 8-19, wherein the patient has a tumor comprising mutation of AKT1 corresponding to position E17, L52, or Q79.

22. The method of any one of claims 8-21, wherein the patient has reduced adverse events (AEs) comparable to a control.

23. The method of claim 22, wherein the patient has reduced severity of one or more AEs selected from the group consisting of fatigue, cough, pain, arthralgia, neutropenia, bradycardia, diarrhea, constipation, dizziness, nausea, anemia, asthenia, thrombocytopenia, or pruritus compared to the control.

24. The method of claim 22, wherein the patient has the same level or reduced level of bradycardia following administration of the combination therapy compared to the control.

25. The method of any one of claims 8-24, wherein the patient has an increased overall survival (OS) comparable to a control.

26. The method of claim 25, wherein the patient has an increased overall survival (OS)of 1, 2, 3, 4, 5, 6, 7, 8, 9, 10, 11, 12, 14, 16, 18, 20, 24 or more months comparable to a control.

27. The method of any one of claims 8-26, wherein duration of response to the combination therapy is increased compared to a control.

28. The method of claim 27, wherein the duration of response is increased by at least 1-3, 2-6, 3-8, 4-10, 5-12, 6-15, 8-20, or 1-24 months.

29. The method of any one of claims 8-28, wherein a patient has increased progression-free survival compared to a control.

30. The method of claim 29, wherein the increase is at least 2, 4, 6, 8, 10, 12, 14, 16, 18, 20, 22, 24, 26, 28, 30, 36, 42, 48, 50, 54, 60, 66, or 72 months.

31. The method any one of claims 22-30, wherein the control is GDC-9545 or a pharmaceutically acceptable salt thereof administered alone ipatasertib or a pharmaceutically acceptable salt thereof administered alone.

32. The method of any one of claims 8-31, wherein the patient has not received prior chemotherapy before administration of the combination therapy.

33. The method of any one of claims 8-31, wherein the patient has been previously treated with tamoxifen.

34. The method of any one of claims 8-31, wherein the patient has been previously treated with a PI3K inhibitor or a mTOR inhibitor prior to administration of the combination therapy.

35. The method of any one of claims 8-31, wherein the patient has not been previously treated with an aromatase inhibitor or a CDK4/6 inhibitor or a combination thereof.

36. A kit comprising the combination therapy of claim 1 and instructions for use.

37. The kit of claim 36, wherein GDC-9545 or a pharmaceutically acceptable salt thereof and ipatasertib or a pharmaceutically acceptable salt thereof are co-formulated.

38. Use of a combination therapy comprising GDC-9545 or a pharmaceutically acceptable salt thereof and ipatasertib or a pharmaceutically acceptable salt thereof for the treatment of IaBC or mBC.

39. Use of a combination therapy comprising GDC-9545 or a pharmaceutically acceptable salt thereof and ipatasertib or a pharmaceutically acceptable salt thereof for the manufacture of a medicament for the treatment of IaBC or mBC.

40. The use of claim 38 or 39, wherein the combination therapy comprises a dosing regimen comprising: (i) administering 30 mg GDC-9545 or a pharmaceutically acceptable salt thereof QD on days 1-28 of a first 28-day cycle; and (ii) administering ipatasertib or a pharmaceutically acceptable salt thereof on days 1-21 of the first 28-day cycle.

41. The use of any one of claims 38-40, wherein the combination therapy is for the treatment of IaBC.

42. The use of any one of claims 38-40, wherein the combination therapy is for the treatment of mBC.

43. A method of inhibiting tumor growth in a patient having IaBC or mBC, the method comprising administering a combination therapy comprising GDC-9545 or a pharmaceutically acceptable salt thereof and ipatasertib or a pharmaceutically acceptable salt thereof in one or more 28-day cycles.

44. A method of producing or improving tumor regression in a patient having IaBC or mBC, the method comprising administering a combination therapy comprising GDC-9545 or a pharmaceutically acceptable salt thereof and ipatasertib or a pharmaceutically acceptable salt thereof in one or more 28-day cycles.
